(19) United States
(12) Patent Application Publication
(10) Pub. No.: US 2003/0036203 A1
(43) Pub. Date: Feb. 20, 2003

(54) Method and graphic representation for profiling oxidative stress and cardiovascular risk

(76) Inventors: Geoff Duke, Phoenix, AZ (US); Russell Duke, Phoenix, AZ (US); Tali Arik, Hobbs, NM (US); Christopher Heward, Redondo Beach, CA (US); Jonathan Thatcher, Scottsdale, AZ (US); Gary Spoleta, Scottsdale, AZ (US); Richard Cutler, Scottsdale, AZ (US)

Correspondence Address: SNELL & WILMER L.L.P., One Arizona Center, 400 East Van Buren, Phoenix, AZ, 85004-2202, US

(21) Appl. No.: 10/027,619

(22) Filed: Oct. 22, 2001

Related U.S. Application Data
(63) Continuation of application No. 09/671,362, filed on Sep. 26, 2000, now abandoned.

(51) Int. Cl. G01N 33/92

(52) U.S. Cl. 436/71; 436/164

(57) ABSTRACT

A method and graphic representation for profiling various risk factors and protective factors relating to an individual's health are presented. The profiles include a bar graph having an optimal value range for at least one indicator substance used for assessing an individual's risk factor or protective factor, where the optimal value range represents optimal health status, as well as a range of values outside of the optimal value range, and an indicator substance value for a test individual or a patient where the test individual or patient indicator substance value is identified on or near the bar graph in relation to the value ranges of indicator substance represented by the bar graph.

### FIELD OF THE INVENTION

The present invention relates generally to a method and graphic representation for profiling various health indicators in order to assist individuals in obtaining optimal health. More particularly, the present invention relates to a graphic display, and a method for devising the same, which represents the extent to which an individual exhibits cardiovascular risk factors, oxidative stress, cancer risk factors, liver function indicators, renal function indicators, age-related hormones and trace metals which are all determined by a plurality of specific assays.

### BACKGROUND OF THE INVENTION

Epidemiological studies have shown that certain factors increase the risk of developing cardiovascular disease. These factors include both genetic and environmental factors as well as factors that may or may not be modifiable.

Traditional risk factors such as, for example, smoking, a diet high in fat and cholesterol, physical inactivity, obesity, low density lipoprotein (LDL) cholesterol, and hypertension have been strongly associated with cardiovascular morbidity and mortality. However, it has been shown that these risk factors can be reduced by changing lifestyle and participating in pharmacologic therapy. More non-traditional and newer cardiovascular risk factors have also been identified such as, for example, chronic inflammation and its markers such as C-reactive protein, homocysteine, oxidative stress or endothelial dysfunction, lipoprotein Lp (a), and psychosocial factors such as environmental stress and responsiveness to stress. Examination of these newer risk factors and their association with cardiovascular disease is just beginning along with their responsiveness to lifestyle modification or pharmacologic therapy.

The risk factors set out above are highly important in evaluating an individual's cardiovascular health. Accordingly, there is a need for a method for graphically representing an individual's cardiovascular health in relation to those risks that can be easily read and interpreted. Moreover, most graphical representations showing cardiovascular risk compare a test subject's values of certain compounds to the values of those compounds that represent the population mean, not the compound values that are associated with optimal cardiovascular health. Therefore, there is also a need to show the graphic representation of a test subject's assay values relating to various risk factors in comparison to those values that are present in an individual having optimal cardiovascular health in order to aid the test subject in achieving optimal cardiovascular health.

Naturally produced free radicals and their relationship to oxidative stress have been the subject of scientific research for the past fifty years. In fact, in the late 1950's, free radicals were observed to increase with the increasing metabolic activity associated with biological oxidation/reduction reactions taking place within the cells of a living body. Accordingly, aging as well as many of the degenerative age-related diseases were suggested to be the result of accumulated free radical damage to cellular constituents and, most particularly, deoxyribonucleic acid (DNA). Therefore, it was concluded that both endogenous and exogenous antioxidants might play an essential role in protecting the body against accumulated free radical damage.

A vast amount of scientific research supports the idea that free radicals are important factors in aging and health. More specifically, scientists have identified a number of specific free radicals or “reactive oxygen species” (ROS), such as superoxide and peroxide, which are toxic by-products of normal energy metabolism. These ROS's are produced in all aerobic cellular reactions in order to produce energy. The steady state level of ROS-mediated damage is called the “oxidative stress status” (OSS) of an individual.

The theory that the accumulation of ROS-mediated damage results in cellular “dysdifferentiation”, or change, is now widely accepted. This cellular dysdifferentiation causes a loss of cell function and a corresponding decline in the functional integrity of the body's organs and tissues. The loss of cell, organ and tissue function are known to have an important causative role in many age-related diseases such as cardiovascular disease, cancer, Alzheimer's disease, and skin disease. The probability of age-related disease is a function of an individual's oxidative stress status (OSS) and, the lower the OSS values, the higher the probability of a long and healthy life.

An individual's OSS is the net result of the rate at which damage is occurring in the body, and the rate at which damage in the body is being cleared. An important factor which determines the rate at which damage occurs in the body is an individual's “antioxidant status” (AOS). Measuring the OSS and AOS of an individual will reveal information about that individual that will enable a physician to design individualized interventions that will work best to decrease the individual's overall oxidative stress, and thereby increase the individual's probability of optimal health. These targeted, individualized interventions involving therapeutics, along with lifestyle changes including diet and exercise, will result in significant improvements in an individual's AOS and OSS.

Several methods and graphic presentations have been utilized to assess oxidative stress. For example, U.S. Pat. No. 5,858,696 discloses a method to assess oxidative stress in vivo by quantifying prostaglandin F₂-like compounds and their metabolites produced by a noncyclooxygenase free radical catalyzed mechanism and comparing them to a control. Further, U.S. Pat. No. 5,891,622 describes a method for assessing oxidative stress in vivo by measuring the amount of free, esterified and glucuronidated forms of isoprostanes (8EPGF2) in a biological sample which contains the isoprostanes and determining the total amount of isoprostanes in the sample. The total isoprostanes amount is then compared with a control sample and oxidative stress is determined where the amount of isoprostanes in the sample increases compared to the control. Likewise, only the glucuronidated form of the isoprostanes may be measured and compared with a control and oxidative stress will be found where the glucuronidated isoprostanes in the sample increase compared to the control.

Another example of a method for determining oxidative stress is described in International Publication Number WO 99/40443 which discloses quantifying the levels or relative distribution of a pair of compounds, o,o′-dityrosine and o-tyrosine, in a patient's urine sample and comparing those levels with the corresponding levels or relative distribution of the same compounds in a normal or control sample. Also, U.S. Pat. No. 5,950,634 discloses another method for assessing oxidative stress in humans which includes preparing a chart where a numerical figure representing the degree of excess or deficiency of an indicator substance indicating oxidative stress is shown in the ordinate and a numerical figure corresponding to the standard value recovered from normal subjects is also shown in the ordinate where the items of the indicator substances are grouped into damaging substances, water-soluble antioxidants, fat-soluble antioxidants, and antioxidant enzymes which are shown in the abscissa. The degree of excess or deficiency of the indicator substance indicating the extent of oxidative stress in humans is collectively shown in the chart by numerically representing the ratio of the measured value of each indicator substance in a test subject to the standard value and recording the resulting numerical figure on the chart.

Although the above referenced prior art shows the measurement and comparison of various compounds to determine oxidative stress, none of the prior art publications discloses the comparison of such compounds in a test subject to an optimal value of the compound which would be present in an individual possessing optimal health status. Instead, the prior art publications compare such compounds in a test individual to the amount of those compounds found present in a normal subject. In other words, in the prior art, the values of the various compounds used to determine oxidative stress in test subjects are compared with values of the compounds which represent the population mean, not values which represent optimal health status.

Further, the prior art to date fails to disclose a method for profiling oxidative stress which utilizes a simple bar graph containing value ranges within and outside of an optimum for specific indicators so that the plotting of a test subject's indicator value can be immediately evaluated as falling within or outside of an optimal range which represents optimal cardiovascular status. Accordingly, there is a need for a clear and simple method for profiling the oxidative stress of a test subject by comparing test subject values relating to oxidative stress to optimal value ranges related to oxidative stress which represent optimal cardiovascular status.

Finally, in that the levels of age-related hormones and trace elements within an individual may also be used to evaluate optimal health, there is also a need for an accurate and clear graphical representation which compares a test subject's levels of age-related hormones and trace elements to those levels seen in individuals possessing optimal health This same type of graphical representation could also be used for illustrating a number of other risk factors including, but not limited to, assay values relating to cancer risk, liver function and renal function.

### BRIEF SUMMARY OF THE INVENTION

It is a principal object of the present invention to provide a method and graphic representation for profiling a patient or individual's risk factors which could lead to less than optimal health for that patient or individual.

It is also an object of the present invention to provide a method and graphic representation for profiling the oxidative stress and/or cardiovascular risk of an individual.

It is another object of the present invention to provide a clear and simple method and graphic representation for profiling an individual's oxidative stress and/or cardiovascular risk which facilitates the recommendation of a customized therapeutic and lifestyle program to achieve the individual's optimal health.

It is yet another object of the present invention to provide a graphic representation, and method for making the same, which presents multiple test results in a format which provides an individual and the individual's physician with the most complete picture possible of the individual's cardiovascular health status.

It is still another object of the present invention to provide a method and graphic representation for profiling an individual's oxidative stress and/or cardiovascular risk based on optimal value ranges of specific, predetermined indicator substances related to oxidative protection, oxidative damage, cardiovascular protection, and/or cardiovascular damage.

In accordance with one exemplary embodiment of the present invention, a method for profiling oxidative stress is presented which includes designating at least one indicator substance for assessing oxidative stress, determining an optimal value range of the indicator that represents optimal cardiovascular status, preparing a bar graph which includes the optimal value range of the indicator substance as well as values outside of the optimal value range, and identifying a value of the indicator substance for an individual by designating the same on or near the bar graph.

In accordance with a further aspect of the present invention, the optimal value range for the indicator substance and the values outside of that optimal range are preferably distinguished from one another by color, symbol, and/or shading. In addition, the bar graph of the present invention may be either horizontal or vertical.

In accordance with yet a further aspect of the present invention, the optimal value range of the indicator substance is determined by calculating an optimal mean value of a defined population which exhibits optimal cardiovascular status.

In accordance with another exemplary embodiment of the present invention, a method for profiling cardiovascular risk is presented which includes designating at least one indicator substance for assessing cardiovascular risk, determining an optimal value range of the indicator that represents optimal cardiovascular status, preparing a bar graph which includes the optimal value range of the indicator substance as well as values outside of the optimal value range, and identifying a value of the indicator substance for an individual or patient by designating the same on or near the bar graph.

In accordance with still another exemplary embodiment of the present invention, a graphic representation for profiling oxidative stress and/or cardiovascular risk is presented which includes a bar graph having an optimal value range for at least one indicator substance for assessing oxidative stress and/or cardiovascular risk which represents optimal cardiovascular status as well as a range of values outside of the optimal value range, and an indicator substance value for an individual or patient that is identified on or near the bar graph in relation to the value ranges represented by the bar graph.

In accordance with still a further aspect of the present invention, the indicator substance, depending upon the particular embodiment of the invention, may relate to cardiovascular protection, cardiovascular damage, oxidative protection, or oxidative damage.

Indicator substances related to cardiovascular protection may include, but are not limited to, apolipoprotein A1, high density lipoproteins, ascorbate, coenzyme Q10, folic acid, alphatocopherol, vitamin B12, transferrin, and iron binding capacity.

Indicator substances related to cardiovascular damage may include, but are not limited to, apolipoprotein B, C reactive protein, cholesterol, glycated protein, homocysteine, iron, iron binding percent saturation, low density lipoproteins, lipoprotein a, fructosamine, ferritin, fibrinogen, and triglycerides. Indicators may also include ratios of the following substances: cholesterol/high density lipoprotein (HDL), Low density lipoprotein (LDL)/HDL, and Apolipoprotein A1/Apolipoprotein A2.

Indicator substances related to oxidative protection may include, but are not limited to, albumin, ascorbate, direct bilirubin, total bilirubin, ceruloplasmin, ferritin, iron binding capacity, selenium, uric acid, glutathione, thiols, coenzyme Q10, alpha and beta carotene, beta cryptoxanthin, lutein, lycopene, retinol, retinol palmitate, zeaxanthin, and tocopherols.

Indicator substances related to oxidative damage may include, but are not limited to, copper, glucose, iron, iron binding percent saturation, ferritin and fructosamine.

It is another object of the present invention to provide an easily interpreted graphical representation for profiling a patient's or test individual's age-related hormones, trace elements, liver indicators, renal indicators, and cancer risk in relation to those same values that are present or should not be present in individuals possessing optimal health.

An individual's PSA may be determined and used as a cancer screen. Liver function indicators may include, but are not limited to, total protein, albumin, globulin, total bilirubin, direct bilirubin, indirect bilirubin, SGOT (AST), SGPT (ALT), GGTP, LDH, and alkaline phosphatase. Renal function indicators may include, but are not limited to, BUN, creatinine, 24 hour urine urea, 24 hour urine protein, 24 hour urine calcium, 24 hour urine creatinine, and creatinine clearance.

Age-related hormones may include, but are not limited to, insulin, DHEA S, cortisol, dihydrotestosterone, estradiol, estriol, estrone, progesterone, SHBG, testosterone, free testost index, thyroid hormones (T3, T4, free T3, free T4, TSH), and IGF-1 (growth hormone). Trace metal values may include, but are not limited to, calcium, iron, magnesium, sodium, potassium, chloride, chromium, cobalt, copper, manganese, molybdenum, selenium, zinc, aluminum, antimony, arsenic, barium, beryllium, cadmium, lead, mercury, nickel, strontium, thallium, and tin.

### BRIEF DESCRIPTION OF THE DRAWINGS

A more complete understanding of the present invention may be derived by referring to the detailed description and claims when considered with the following illustrative Figures, which may not be to scale. In the following Figures, like reference numbers refer to similar elements throughout the Figures.

FIG. 1 is a graphic representation profiling a single indicator substance which relates to a risk factor or a protective factor in determining optimal health;

FIG. 2 is a graphic representation profiling cardiovascular risk in accordance with one exemplary embodiment of the present invention;

FIG. 3 is a graphic representation profiling oxidative stress in accordance with another exemplary embodiment of the present invention;

FIG. 4 is a graphic representation profiling a cancer screen indicator in accordance with another exemplary embodiment of the present invention;

FIG. 5 is a graphic representation profiling liver function indicators in accordance with another exemplary embodiment of the present invention;

FIG. 6 is a graphic representation profiling renal function indicators in accordance with another exemplary embodiment of the present invention;

FIGS. 7-12 are graphic representations profiling age-related hormones in accordance with another embodiment of the present invention;

FIGS. 13-15 are graphic representations profiling trace metals in accordance with another embodiment of the present invention; and

FIG. 16 is a flow chart depicting a method for profiling a single indicator substance which relates to a risk factor or a protective factor in determining optimal health.

### DETAILED DESCRIPTION OF EXEMPLARY EMBODIMENTS

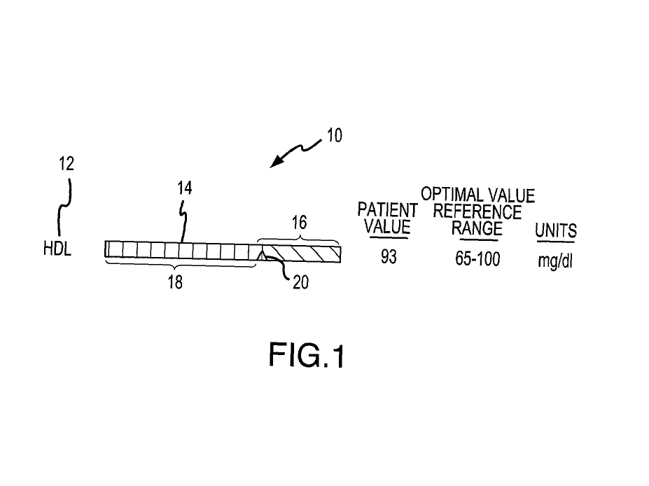

It should be appreciated that the particular embodiments shown and described herein are illustrative of the invention and its best mode and are not intended to otherwise limit the scope of the present invention in any way. FIG. 1 shows a graphic representation 10 for a profile of a single risk factor or protective factor 12 in determining optimal health for a patient or individual. For exemplary purposes, the risk factor or protective factor 12 is represented by the protective factor high density lipoprotein (HDL) which may be used as an indicator substance in evaluating a patient's or individual's oxidative stress and/or cardiovascular risk. The graphic representation 10 includes a bar graph 14 which has both an optimal value range 16 for the protective factor 12, in this case HDL, as well as a range of values outside the optimal range 18, and a patient value or individual value 20 for the protective factor 12 which is identified or marked on or near the bar graph 14 in accordance with the range of values represented by the bar graph 14.

The optimal value range 16 and the range of values outside the optimal value range 18 are preferably distinguished from one another by color, symbol, shading, or any other similar means that is available to distinguish different value ranges contained within a bar graph. In FIG. 1, the range of values which exist outside of the optimal value range 18 are represented by hatched areas of the bar graph in order to distinguish this set of values from the optimal value range 16 which is represented by clear areas on the bar graph. Preferably, the optimal value range 16 and the values outside of the optimal value range 18 are distinguished by color, the color green for areas of the bar graph 14 falling within the optimal value range 16 and the color red for areas of the bar graph 14 falling outside of the optimal range 18, so that it can be easily determined as to whether the patient's or individual's value for the risk factor or protective factor falls within or outside of the optimal value range for that risk factor or protective factor once the individual's value for the risk factor or protective factor is identified or marked on the bar graph 14.

The optimal value range 16 for each risk factor or protective factor 12 is determined by calculating an optimal mean value of the risk factor or protective factor 12 in a defined population which exhibits optimal health. The risk factor or protective factor usually comprises an indicator substance or compound while the optimal mean may be based on overall optimal health, optimal cardiovascular health, optimal reproductive health, optimal digestive health, etc. This determination of optimal value range 16 for a risk factor or protective factor 12 differs from other acceptable value ranges for such factors or indicator substances which are used in assessing optimal health in that the optimal value range 16 represents the value range of a risk factor or protective factor that is present in those individuals with optimal health. In contrast, an “acceptable” value range of a risk factor or protective factor used for assessing health conditions such as oxidative stress and/or cardiovascular risk is determined by calculating the mean value of the risk factor or protective factor which is present in a normal population of individuals, i.e. those individuals whose cardiovascular status ranges from average to optimal. Accordingly, the optimal value range 16 for risk factors and protective factors in the present invention used to assess health conditions or parameters such as oxidative stress and/or cardiovascular risk reflect values consistent with optimum health and are thereby targeted to efficiently move an individual to a status of optimum health rather than average health.

Individual values for risk factors and protective factors are determined by taking blood and/or urine samples from the patient or individual and assaying those samples for the amount of risk factors or protective factors present in the sample. In the case of the protective factor HDL 12 shown in FIG. 1, it can be seen that the optimal value range 16 for HDL in those individuals exhibiting optimal cardiovascular status ranges from 65 to 100 milligrams per deciliter and that the patient's or individual's value 20 for the protective factor in this instance is 98 milligrams per deciliter which is shown to fall within the optimal value range 16 for HDL.

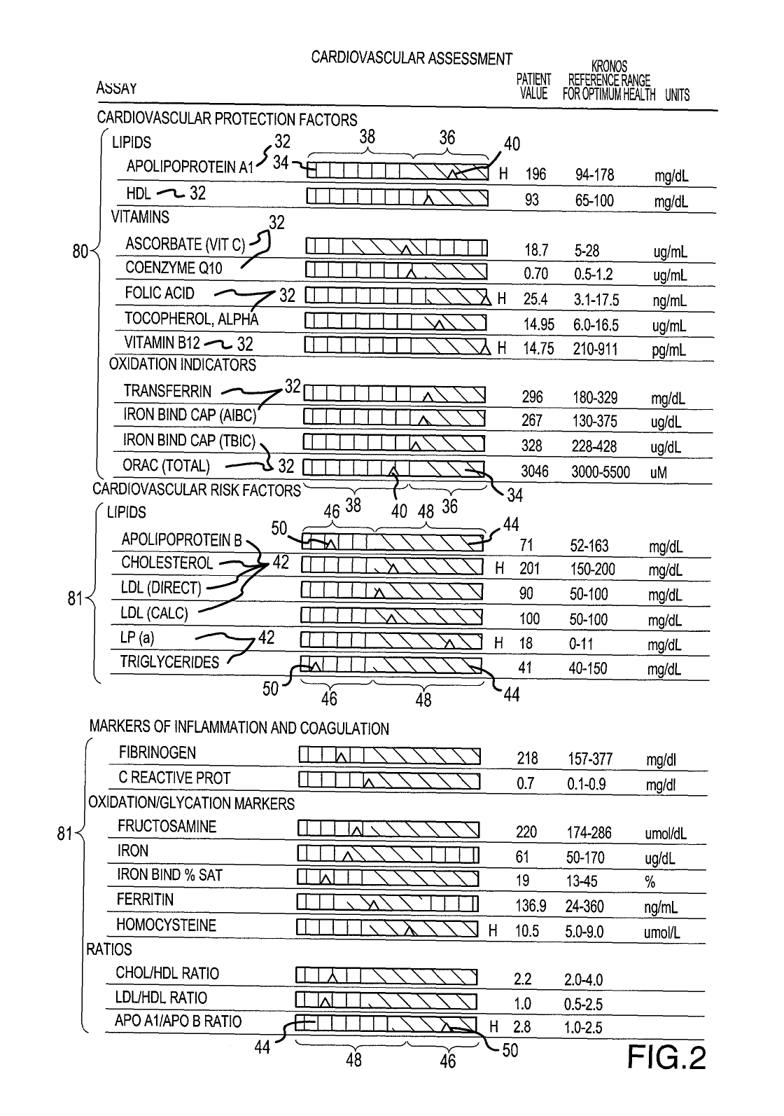

FIG. 2 shows a graphic representation profiling cardiovascular risk in accordance with one exemplary embodiment of the present invention. The graphic representation in FIG. 2 includes two profile sets, one for assessing cardiovascular protection 30 and one for assessing cardiovascular damage 31. The profile set for assessing cardiovascular protection 30 includes individual bar graphs 34 which profile a plurality of protective factors (also referred to as indicator substances) 32 that are related to cardiovascular protection. These indicator substances related to cardiovascular protection shown in FIG. 2 include, but are not limited to, apolipoprotein A1, HDL, ascorbate, coenzyme Q10, folic acid, tocopherol, vitamin B12, transferrin, iron binding capacity, and total ORAC. Individual graphic representations profiling each of these protective factors are shown using individual bar graphs 34 for each protective factor. The profile for each individual protective factor 32 includes a bar graph having an optimal value range 36 for the protective factor 32 as well as a range of values outside of the optimal range 38 for the protective factor 32, and a patient's or individual's value 40 for the protective factor 32 which is identified or marked on or near the bar graph 34 in relation to the range of values represented by the bar graph 34. As a result, both the patient or individual and their physician can easily identify those protective factors for the patient or individual which fall outside of the optimal value range. Both the patient or individual and their physician can then decide appropriate interventions and/or therapies for optimizing health by choosing those interventions and/or therapies that will move a patient's or individual's protective factor values from outside of the optimal value range back into the optimal value range. The optimal value ranges for the protective factors relating to cardiovascular protection shown in FIG. 2 are contained in Table I.

TABLE I

| Protective Factors (Indicator Substances) | Optimal |
|---|---|
| Relating to Cardiovascular Protection | Value Reference Range |
| apolipoprotein A1 | 94-178 mg/dL |
| HDL | 65-100 mg/dL |
| ascorbate | 5-28 mg/mL |
| alphatocopherol | 6.0-16.5 mg/mL |
| vitamin B12 | 210-911 pg/mL |
| transferrin | 180-329 mg/dL |
| available iron binding capacity (AIBC) | 130-375 mg/dL |
| total iron binding capacity (TIBC) | 228-428 mg/dL |
| ORAC (total) | 3000-5500 uM |

The profile set for assessing cardiovascular damage 31 includes individual bar graphs 44 which profile a plurality of risk factors (also referred to as indicator substances) 42 that are related to cardiovascular damage. These risk factors related to cardiovascular damage shown in FIG. 2 include, but are not limited to, apolipoprotein B, cholesterol, direct LDL, LDL(calc), lipoprotein (a) triglycerides, fibrinogen, C reactive protein, fructosamine, iron, iron binding % saturation, ferritin, homocysteine, cholesterol/HML ratio, LDL/HDL ratio, and apolipoprotein A1/apolipoprotein B ratio. Individual graphic representations profiling each of these risk factors are shown using individual bar graphs 44 for each risk factor. The profile for each individual risk factor 42 includes a bar graph having an optimal value range 46 for the risk factor 42 as well as a range of values outside of the optimal range 48 for the risk factor 42, and a patient's or individual's value 50 for the risk factor 42 which is identified or marked on or near the bar graph 44 in relation to the range of values represented by the bar graph 44. As a result, both the patient or individual and their physician can easily identify those risk factors for the patient or individual which fall outside of the optimal value range. Both the patient or individual and their physician can then decide appropriate interventions and/or therapies for optimizing health by choosing those interventions and/or therapies that will move a patient's or individual's risk factor values from outside of the optimal value range back into the optimal value range. The optimal value ranges for the risk factors relating to cardiovascular damage shown in FIG. 2 are contained in Table II.

TABLE II

| Risk Factors (Indicator Substances) | Optimal |
|---|---|
| Relating to Cardiovascular Damage | Value Reference Range |
| apolipoprotein B | 52-163 mg/dL |
| cholesterol | 150-200 mg/dL |
| direct LDL | 50-100 mg/dL |
| LDL (calc) | 50-100 mg/dL |
| lipoprotein (a) | 0-11 mg/dL |
| triglycerides | 40-150 mg/dL |
| fibrinogen | 157-377 mg/dL |
| C reactive protein | 0.1-0.9 mg/dL |
| fructosamine | 174-286 umol/dL |
| iron | 50-170 mg/dL |
| iron binding % saturation | 13-45% |
| ferritin | 24-360 ng/mL |
| homocysteine | 5.0-9.0 umol/L |
| cholesterol/HDL ratio | 2.0-4.0 |
| LDL/HDL ratio | 0.5-2.5 |
| apolipoprotein A1/apolipoprotein B ratio | 1.0-2.5 |

The probability of age-related disease is a function of an individual's oxidative stress status (OSS). The lower an individual's OSS, the higher the probability of a long and healthy life. An individual's OSS is the net result of the rate at which damage is occurring in the body, and the rate at which damage is being cleared. An individual's “antioxidant status” (AOS) is an important factor in determining the rate at which damage occurs in an individual. By measuring an individual's OSS and AOS, a physician is able to design custom interventions that will best decrease an individual's overall OSS thereby increasing the probability of optimal health.

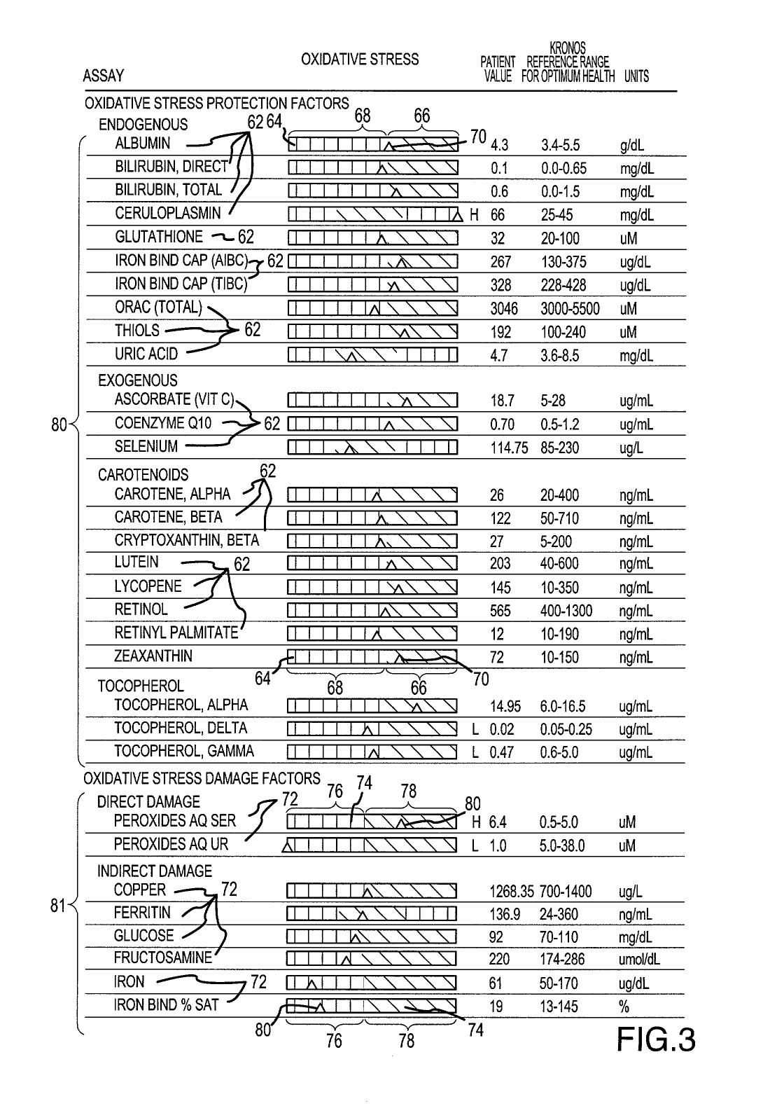

The graphic representation for profiling oxidative stress in the present invention graphically displays those factors related to oxidative protection and oxidative damage. FIG. 3 shows a graphic representation profiling oxidative stress in accordance with one exemplary embodiment of the present invention. The graphic representation in FIG. 3 includes two profile sets, one for assessing oxidative protection 60 and one for assessing oxidative damage 61. The profile set for assessing oxidative protection 60 includes individual bar graphs 64 which profile a plurality of protective factors (also referred to as indicator substances) 62 that are related to oxidative protection. These protective factors related to oxidative protection shown in FIG. 3 include, but are not limited to, albumin, direct bilirubin, total bilirubin, ceruloplasmin, glutathione, available iron binding capacity (AIBC), total iron binding capacity (TIBC), total ORAC, thiols, uric acid, ascorbate, coenzyme Q10, selenium, alpha carotene, beta carotene, beta cryptoxanthin, lutein, lycopene, retinol, retinyl palmitate, zeaxanthin, alpha tocopherol, delta tocopherol, and gamma tocopherol. Individual graphic representations profiling each of these protective factors are shown using individual bar graphs 64 for each protective factor. The profile for each protective factor 62 includes a bar graph having an optimal value range 66 for the protective factor 62 as well as a range of values outside of the optimal range 68 for the protective factor 62, and a patient's or individual's value 70 for the protective factor 62 which is identified or marked on or near the bar graph 64 in relation to the range of values represented by the bar graph 64. As a result, both the patient or individual and their physician can easily identify those protective factors for the patient or individual which fall outside of the optimal value range. Both the patient or individual and their physician can then decide appropriate interventions and/or therapies for optimizing health by choosing those interventions and/or therapies that will move a patient's or individual's protective factor values from outside of the optimal value range back into the optimal value range. The optimal value ranges for the protective factors relating to oxidative protection shown in FIG. 3 are contained in Table III.

TABLE III

| Protective Factors (Indicator Substances) | Optimal |
|---|---|
| Relating to Oxidative Protection | Value Reference Range |
| albumin | 3.4-5.5 g/dL |
| direct bilirubin | 0.0-0.65 mg/dL |
| total bilirubin | 0.0-1.5 mg/dL |
| ceruloplasmin | 25-45 mg/dL |
| glutathione | 20-100 7M |
| available iron binding capacity (AIBC) | 130-375 ug/dL |
| total iron binding capacity (TIBC) | 228-428 ug/dL |
| total ORAC | 3000-5500 uM |
| thiols | 100-240 uM |
| uric acid | 3.6-8.5 mg/dL |
| ascorbate | 5-28 ug/mL |
| coenzyme Q10 | 0.5-1.2 ug/mL |
| selenium | 85-230 ug/L |
| alpha carotene | 20-400 ng/mL |
| beta carotene | 50-710 ng/mL |
| beta cryptoxanthin | 5-200 ng/mL |
| lutein | 40-600 ng/mL |
| lycopene | 10-350 ng/mL |
| retinol | 400-1300 ng/mL |
| retinyl palmitate | 10-190 ng/mL |
| zeaxanthin | 10-150 ng/mL |
| alpha tocopherol | 6.0-16.5 ug/mL |
| delta tocopherol | 0.05-0.25 ug/mL |
| gamma tocopherol | 0.6-5.0 ug/mL |

The profile set for assessing oxidative damage 61 includes individual bar graphs 74 which profile a plurality of risk factors (also referred to as indicator substances) 72 that are related to oxidative damage. These risk factors related to oxidative damage shown in FIG. 3 include, but are not limited to, peroxides, copper, ferritin, glucose, fructosamine, iron, and iron binding % saturation. Individual graphic representations profiling each of these risk factors are shown using individual bar graphs 74 for each risk factor. The profile for each individual risk factor 72 includes a bar graph having an optimal value range 76 for the risk factor 72 as well as a range of values outside of the optimal range 78 for the risk factor 72, and a patient's or individual's value 80 for the risk factor 72 which is identified or marked on or near the bar graph 74 in relation to the range of values represented by the bar graph 74. As a result, both the patient or individual and their physician can easily indicate those risk factors for the patient or individual which fall outside of the optimal value range. Both the patient or individual and their physician can then decide appropriate interventions and/or therapies for optimizing health by choosing those interventions and/or therapies that will move a patient's or individual's risk factor values from outside of the optimal value range back into the optimal value range. The optimal value ranges for the risk factors relating to oxidative damage shown in FIG. 3 are contained in Table IV.

TABLE IV

| Risk Factors (Indicator Substances) | |
|---|---|
| Relating to Oxidative Damage | Optimal Value Reference Range |
| peroxides AQ serum | 5.0-5.0 uM |
| peroxides AQ urine | 5.0-38.0 uM |
| copper | 700-1400 ug/L |
| ferritin | 24-360 ng/mL |
| glucose | 70-110 mg/dL |
| fructosamine | 174-286 umol/dL |
| iron | 50-170 ug/dL |
| iron binding % saturation | 13-45% |

The comprehensive oxidative stress assessment report serves as a basis for a physician's recommendation of a customized therapeutic program developed specifically for a patient that is unique to that patient. A large number of sensitive and specific assays, such as those for detecting and measuring the indicator substances described above, form the basis for close monitoring and follow-up of the results from a regimen of vitamins, minerals, hormones, and other pharmaceuticals which may be specifically designed for a patient or individual. A physician can regularly recalibrate a specific individual's regimen by periodically repeating the assays relating to oxidative stress so that optimal health can be achieved and maintained.

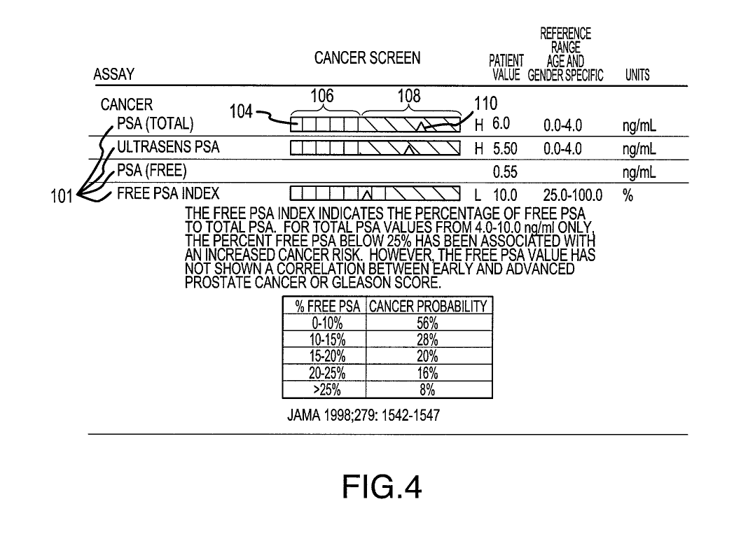

Turning now to FIG. 4, a profile utilizing PSA for a cancer screen is shown. The indicator substances 101 used to conduct the cancer screen include total PSA, ultrasens PSA, and free PSA index. The profile set for the cancer screen includes individual bar graphs 104 which profile a plurality of indicator substances 101 that are related to the cancer screen. Individual graphic representations profiling each of these indicator substances are shown using individual bar graphs 104 for each indicator substance 101. The profile for each individual indicator substance 101 includes a bar graph having an optimal value range 106 for the indicator substance 101 as well as a range of values outside of the optimal value range 108 for the indicator substance 101 and a patient's or individual's value 110 for the indicator substance 101 which is identified or marked on or near the bar graph 104 in relation to the range of values represented by the bar graph 104. As a result, both the patient or individual and their physician can easily identify those indicator substances for the patient or individual which fall outside of the optimal value range. Both the patient or individual and their physician can then decide appropriate interventions and/or therapies for optimizing health by choosing those interventions and/or therapies that will move a patient's or individual's indicator substance values from outside of the optimal value range back into the optimal value range. The optimal value ranges for the indicator substances relating to the cancer screen shown in FIG. 4 are contained in Table V.

TABLE V

| Indicator Substance for Cancer Screen | Optimal Value Reference Range |
|---|---|
| total PSA | 0.0-4.0 ng/mL |
| ultrasens PSA | 0.0-4.0 ng/mL |
| free PSA index | 25-100% |

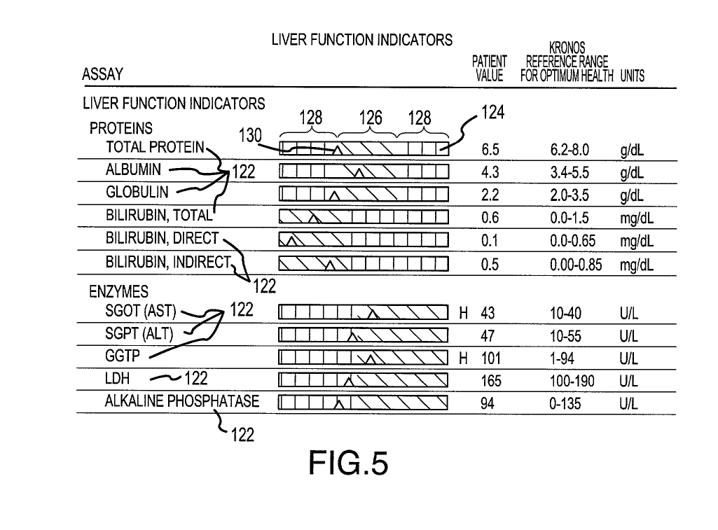

FIG. 5 shows a graphic representation profiling liver function indicators in accordance with another exemplary embodiment of the present invention. The graphic representation in FIG. 5 includes a profile set for proteins and a profile set for enzymes. The profile set for assessing liver function indicators 122 includes individual bar graphs 124 which profile a plurality of indicator substances 122 that are related to liver function. These indicator substances related to liver function shown in FIG. 5 include, but are not limited to, total protein, albumin, globulin, total bilirubin, direct bilirubin, indirect bilirubin, SGOT (AST), SGPT (ALT), GGTP, LDH, and alkaline phosphatase. Individual graphic representations profiling each of these indicator substances are shown using individual bar graphs 124 for each indicator substance 122. The profile for each individual indicator substance 122 includes a bar graph having an optimal value range 126 for the indicator substance 122 as well as a range of values outside of the optimal range 128 for the indicator substance 122, and a patient's or individual's value 130 for the indicator substance 122 which is identified or marked on or near the bar graph 124 in relation to the range of values represented by the bar graph 124. As a result, both the patient or individual and their physician can easily identify those liver function indicators for the patient or individual which fall outside of the optimal value range. Both the patient or individual and their physician can then decide appropriate interventions and/or therapies for optimizing health by choosing those interventions and/or therapies that will move a patient's or individual's liver function indicator values from outside of the optimal value range back into the optimal value range. The optimal value ranges for the liver function indicators shown in FIG. 5 are contained in Table VI.

TABLE VI

| Liver Function Indicators | Optimal Value Reference Range |
|---|---|
| total protein | 6.2-8.2 g/dL |
| albumin | 3.4-5.5 g/dL |
| globulin | 2.0-3.5 g/dL |
| total bilirubin | 0.0-1.5 mg/dL |
| direct bilirubin | 0.0-0.65 mg/dL |
| indirect bilirubin | 0.0-0.85 mg/dL |
| SGOT (AST) | 10-40 U/L |
| SGPT (ALT) | 10-55 U/L |
| GGTP | 1-94 U/L |
| LDH | 100-190 U/L |
| alkaline phosphatase | 0-135 U/L |

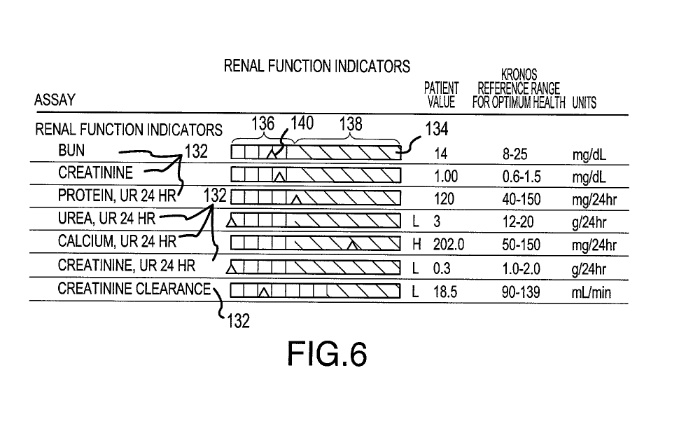

The profile set for assessing renal function is shown in FIG. 6. The profile set for assessing renal function includes individual bar graphs 134 which profile a plurality of renal function indicators 132 that are related to renal function. These indicator substances related to renal function shown in FIG. 6 include, but are not limited to, BUN, creatinine, 24-hr. urine protein, 24-hr. urine urea, 24-hr. urine calcium, 24-hr. urine creatinine, and creatinine clearance. Individual graphic representations profiling each of these renal function indicators are shown using individual bar graphs 134 for each indicator substance. The profile for each individual indicator substance 132 includes a bar graph having an optimal value range 136 for the indicator substance 132 as well as a range of values outside of the optimal range 138 for the indicator substance 132, and a patient's or individual's value 140 for the indicator substance 132 which is identified or marked on or near the bar graph 134 in relation to the range of values represented by the bar graph 134. As a result, both the patient or individual and their physician can easily identify those renal function indicators for the patient or individual which fall outside of the optimal value range and determine appropriate interventions and/or therapies for optimizing health by choosing those interventions and/or therapies that will move a patient's or individual's renal function indicator values from outside of the optimal value range back into the optimal value range. The optimal value ranges for the renal function indicators shown in FIG. 6 are contained in Table VII.

TABLE VII

| Renal Function Indicators | Optimal Value Reference Range |
|---|---|
| BUN | 8-25 mg/dL |
| creatinine | 0.6-1.5 mg/dL |
| urine protein, 24 hrs. | 40-150 mg/24 hr |
| urine urea, 24 hrs. | 12-20 g/24 hr |
| urine calcium, 24 hrs. | 50-150 mg/24 hr |
| urine creatinine, 24 hrs. | 1.0-2.0 g/24 hr |
| creatinine clearance | 90-139 mL/min |

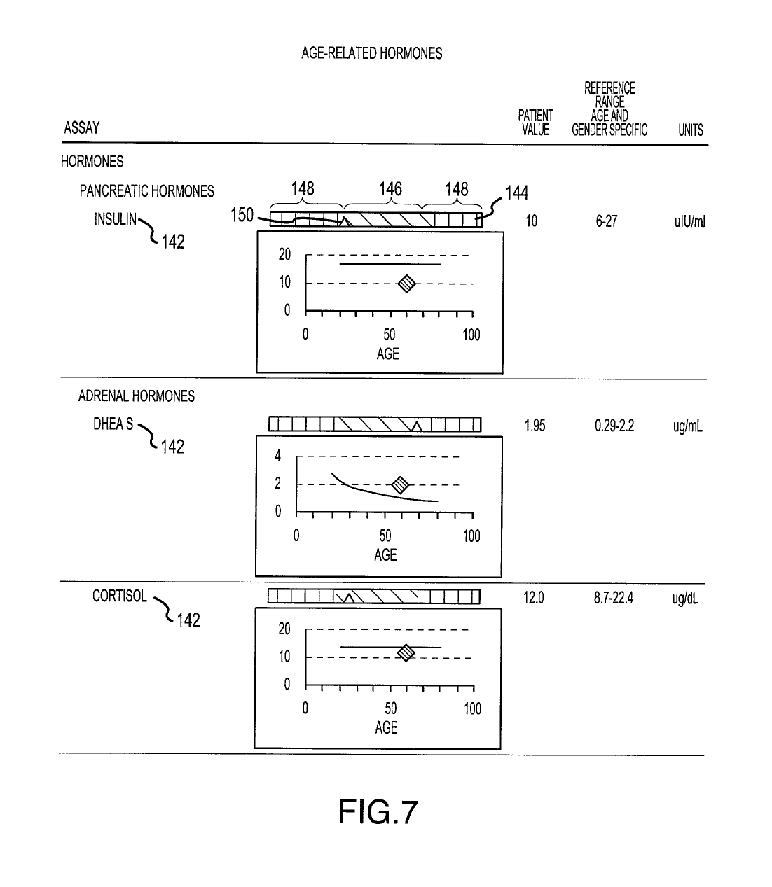

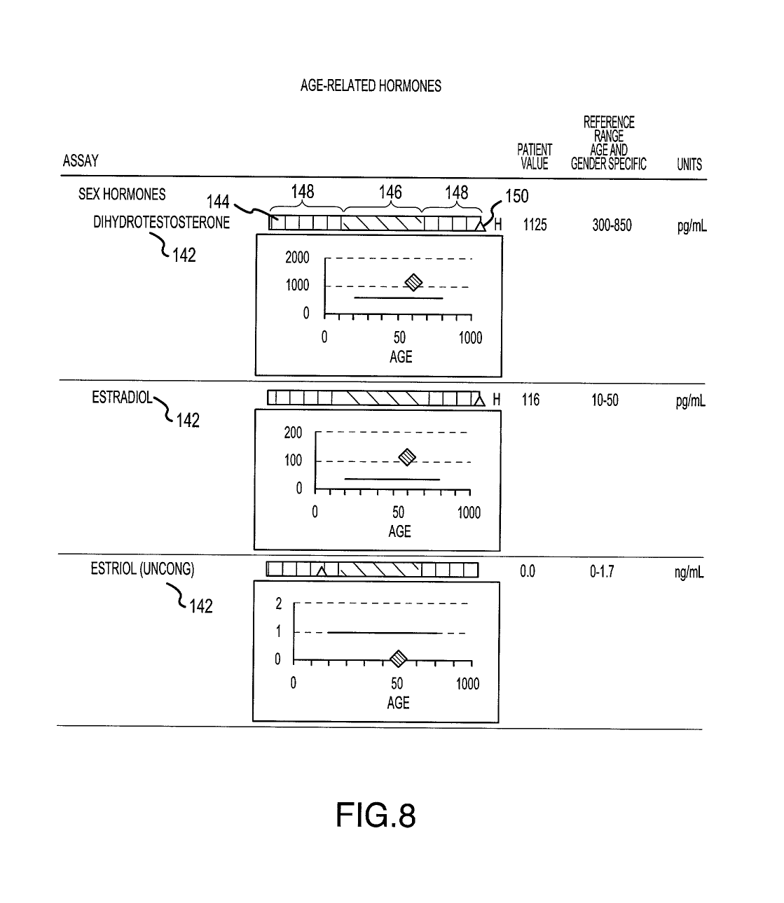

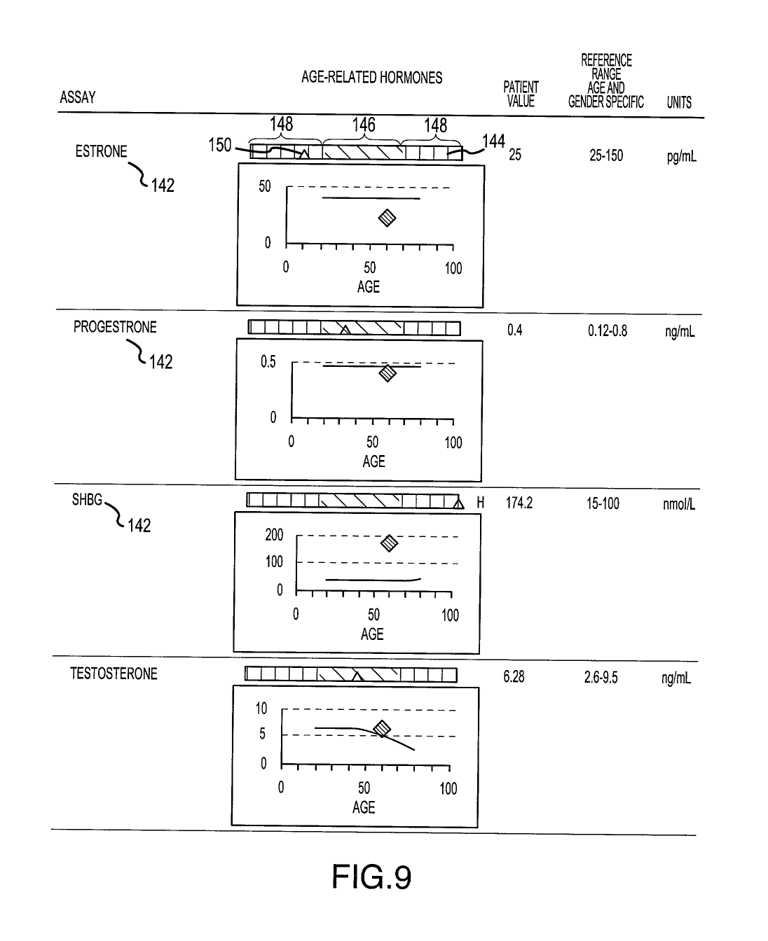

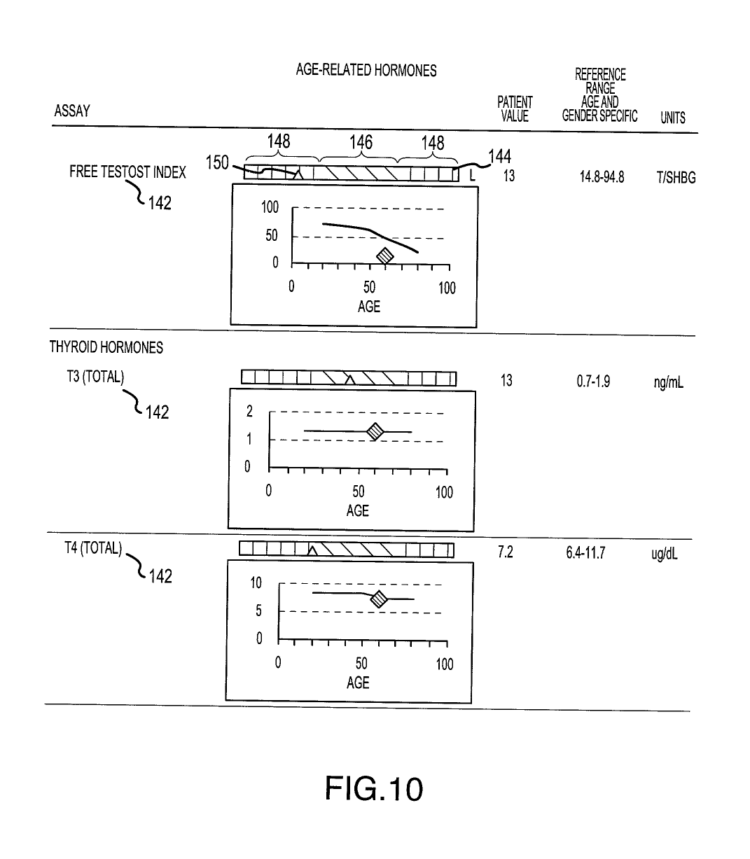

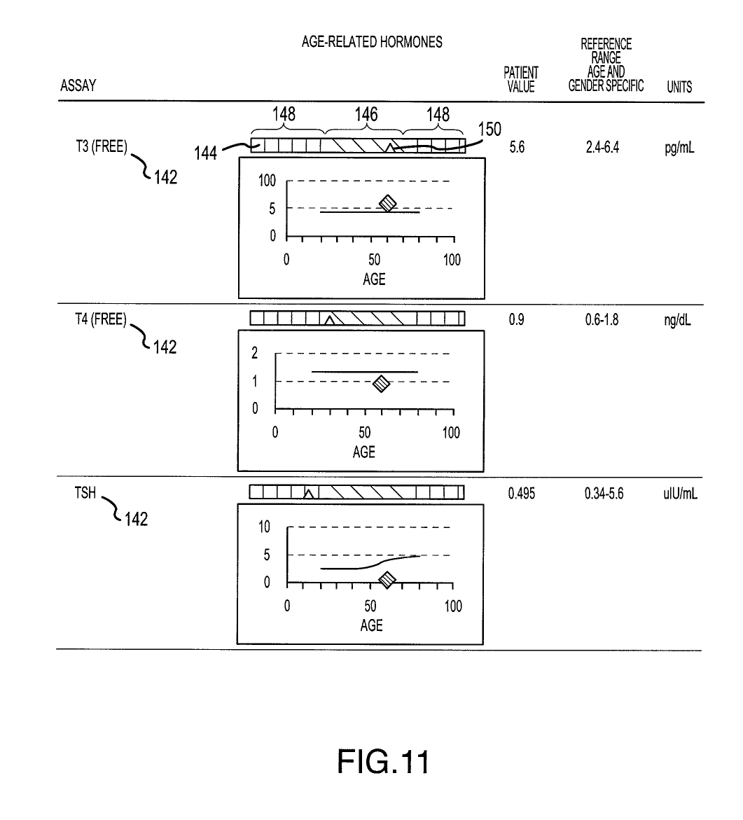

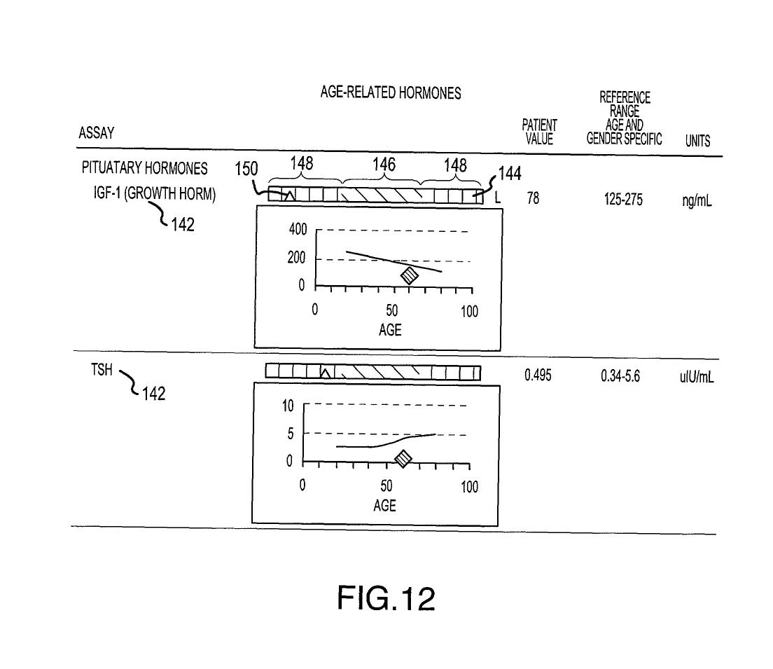

FIGS. 7-12 show the profile set for age-related hormones. The profile set for age-related hormones 142 includes individual bar graphs 144 which profile a plurality of age-related hormones 142. These age-related hormones shown in FIGS. 7-12 include, but are not limited to, insulin, DHEA S, cortisol, dihydrotestosterone, estradiol, estriol (uncong), estrone, progesterone, SHBG, testosterone, free testosterone index, total T3, total T4, free T3, free T4, TSH thyroid hormones, IGF-1 growth hormone, and TSH pituitary hormones. Individual graphic representations profiling each of these age-related hormones are shown using individual bar graphs 144 for each age-related hormone 142. The profile for each individual age-related hormone 142 includes a bar graph having an optimal value range 146 for the age-related hormone 142 as well as a range of values outside of the optimal range 148 for the age-related hormone 142, and a patient's or individual's value 150 for the age-related hormone 142 which is identified or marked on or near the bar graph 144 in relation to the range of values represented by the bar graph 144. Accordingly, both the patient or individual and their physician can easily identify those age-related hormones for the patient or individual which fall outside of the optimal value range and can then address choosing those interventions and/or therapies that will move a patient's or individual's age-related values from outside of the optimal value range back into the optimal value range. The optimal value ranges for the age-related hormones shown in FIGS. 7-12 are contained in Table VIII.

TABLE VIII

| Age-Related Hormones | Optimal Value Reference Range |
|---|---|
| insulin | 6-27 ulU/mL |
| DHEA S | 0.29-2.2 ug/mL |
| cortisol | 8.7-22.4 ug/dL |
| dihydrotestosterone | 300-850 pg/mL |
| estradiol | 10-50 pg/mL |
| estriol (uncong) | 0-1.7 ng/mL |
| estrone | 25-150 pg/mL |
| progesterone | 0.12-0.8 ng/mL |
| SHBG | 15-100 nmol/L |
| testosterone | 2.6-9.5 ng/mL |
| free testosterone index | 14.8-94.8 T/SHBG |
| total T3 | 0.7-1.9 ng/mL |
| total T4 | 6.4-11.7 ug/dL |
| free T3 | 2.4-6.4 pg/mL |
| free T4 | 0.6-1.8 ng/dL |
| TSH thyroid hormones | 0.34-5.6 ulU/mL |
| IGF-1 (growth hormone) | 125-275 ng/mL |
| TSH pituitary hormones | 0.34-5.6 ulU/mL |

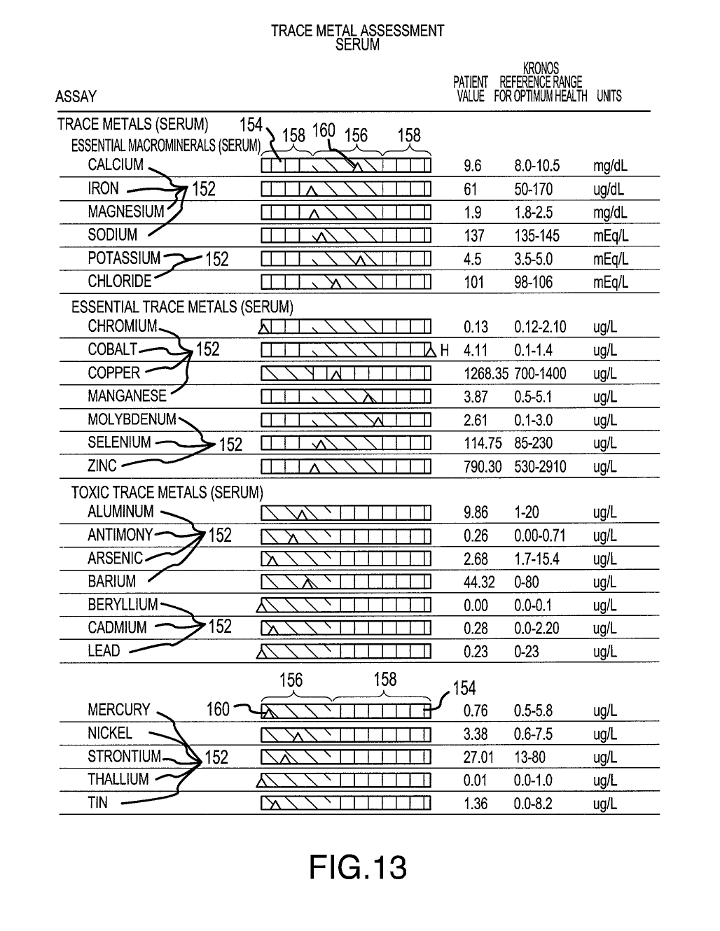

Tables XIII-XV show serum trace metal profiles for essential macrominerals, essential trace metals, and toxic trace metals. Individual graphic representations profiling each of these serum trace metals are shown using individual bar graphs 154 for each trace metal 152. The profile for each individual serum trace metal 152 includes a bar graph having an optimal value range 156 for the trace metal 152 as well as a range of values outside of the optimal range 158 for the serum trace metal 152, and a patient's or individual's value 160 for the serum trace metal 152 which is identified or marked on or near the bar graph 154 in relation to the range of values represented by the bar graph 154. As with the other indicator substances, both the patient or individual and their physician can easily identify those serum trace metals for the patient or individual which fall outside of the optimal value range and can then determine appropriate interventions and/or therapies for moving those trace metal values back into the optimal value range. The optimal value ranges for the serum trace metals shown in FIG. 13 are contained in Table IX.

TABLE IX

| Serum Trace Metals | Optimal Value Reference Range |
|---|---|
| calcium | 8.0-10.5 mg/dL |
| iron | 50-170 ug/dL |
| magnesium | 1.8-2.5 mg/dL |
| sodium | 135-145 mEq/L |
| potassium | 3.5-5.0 mEq/L |
| chloride | 98-106 mEq/L |
| chromium | 0.12-2.10 ug/L |
| cobalt | 0.1-1.4 ug/L |
| copper | 700-1400 ug/L |
| manganese | 0.5-5.1 ug/L |
| molybdenum | 0.1-3.0 ug/L |
| selenium | 85-230 ug/L |
| zinc | 530-2,910 ug/L |
| aluminum | 1-20 ug/L |
| antimony | 0.00-0.71 ug/L |
| arsenic | 1.7-15.4 ug/L |
| barium | 0-80 ug/L |
| beryllium | 0.0-0.1 ug/L |
| cadmium | 0.0-2.20 ug/L |
| lead | 0-23 ug/L |
| mercury | 0.5-5.8 ug/L |
| nickel | 0.6-7.5 ug/L |
| strontium | 13-80 ug/L |
| thallium | 0.0-1.0 ug/L |
| tin | 0.0-8.2 ug/L |

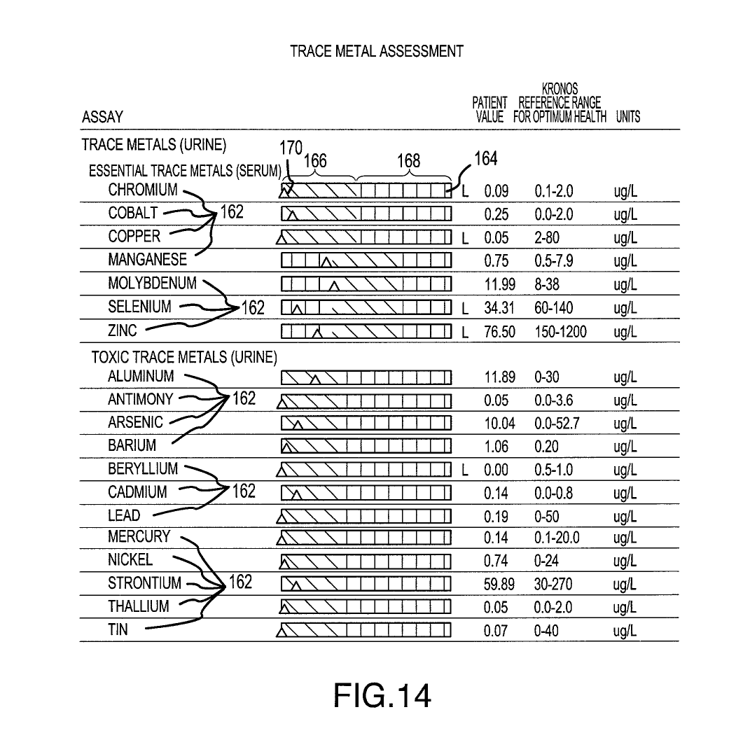

FIG. 14 shows the profiles of individual trace metals 162 for urine trace metal assessment. Individual graphic representations profiling each of these urine trace metals are shown using individual bar graphs 164 for each trace metal 162. The profile for each urine trace metal 162 includes a bar graph having an optimal value range 166 for the trace metal 162 as well as a range of values outside of the optimal value range 168 for the indicator substance 162, and a patient's or individual's value 170 for the urine trace metal 162 which is identified or marked on or near the bar graph 164 in relation to the range of values represented by the bar graph 164. The patient or individual and their physician can easily identify the patient's or individual's urine trace metal values which fall outside of the optimal value range and can then work to manipulate the patient's or individual's value by determining appropriate interventions and/or therapies for optimizing health by choosing those interventions and/or therapies that will move the patient's or individual's urine trace metal value back into the optimal value range. Optimal value ranges for urine trace metals shown in FIG. 14 are contained in Table X.

TABLE X

| Urine Trace Metals | Optimal Value Reference Range |
|---|---|
| chromium | 0.1-2.0 ug/L |
| cobalt | 0.0-2.0 ug/L |
| copper | 2-80 ug/L |
| manganese | 0.5-7.9 ug/L |
| molybdenum | 8-38 ug/L |
| selenium | 60-140 ug/L |
| zinc | 150-1200 ug/L |
| aluminum | 0-30 ug/L |
| antimony | 0.0-3.6 ug/L |
| arsenic | 0.0-52.7 ug/L |
| barium | 0-20 ug/L |
| beryllium | 0.5-1.0 ug/L |
| cadmium | 0.0-0.8 ug/L |
| lead | 0-50 ug/L |
| mercury | 0.1-20.0 ug/L |
| nickel | 0-24 ug/L |
| strontium | 30-270 ug/L |
| thallium | 0.0-2.0 ug/L |
| tin | 0-40 ug/L |

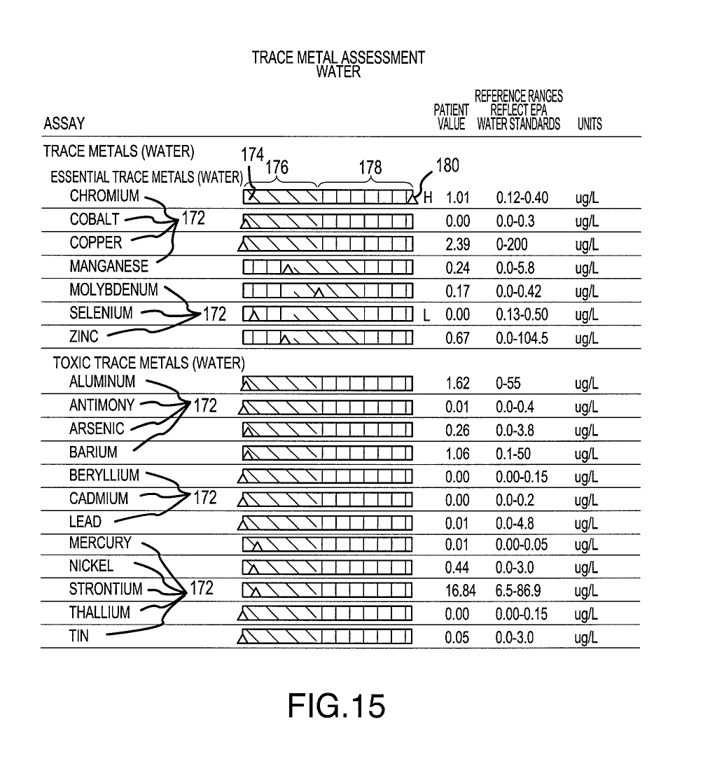

Trace metals found in an individual's water supply are profiled in FIG. 15. FIG. 15 shows individual graphic representations profiling trace metals present in an individual's water source are shown using individual bar graphs 174 for each trace metal 172. The profile for each trace metal 172 found in an individual's water supply includes a bar graph having an optimal value range 176 for the trace metal 172 as well as a range of values outside of the optimal range 178 for the trace metal 172, and a patient's or individual's value 180 for the water trace metal 172 which is identified or marked on or near the bar graph 174 in relation to the range of values represented by the bar graph 174. Again, as with the previously described trace metal assessments, a patient or individual and their physician may then easily determine which trace metals within a patient's or individual's water supply fall outside of the optimal value range. The patent or individual and physician can then decide appropriate interventions in order to move the patient's or individual's water trace metal value back into the optimal value range. The optimal value ranges for the trace metals found in an individual's water supply shown in FIG. 15 are contained in Table XI.

TABLE XI

| Trace Metals in Water Supply | Optimal Value Reference Range |
|---|---|
| chromium | 0.12-0.40 ug/L |
| cobalt | 0.0-0.3 ug/L |
| copper | 0-200 ug/L |
| manganese | 0.0-5.8 ug/L |
| molybdenum | 0.0-0.42 ug/L |
| selenium | 0.13-0.50 ug/L |
| zinc | 0.0-104.5 ug/L |
| aluminum | 0-55 ug/L |
| antimony | 0.0-0.4 ug/L |
| arsenic | 0.0-3.8 ug/L |
| barium | 0.1-50 ug/L |
| beryllium | 0.00-0.15 ug/L |
| cadmium | 0.0-0.2 ug/L |
| lead | 0.0-4.8 ug/L |
| mercury | 0.00-0.05 ug/L |
| nickel | 0.0-3.0 ug/L |
| strontium | 6.5-86.9 ug/L |
| thallium | 0.00-0.15 ug/L |
| tin | 0.0-3.0 ug/L |

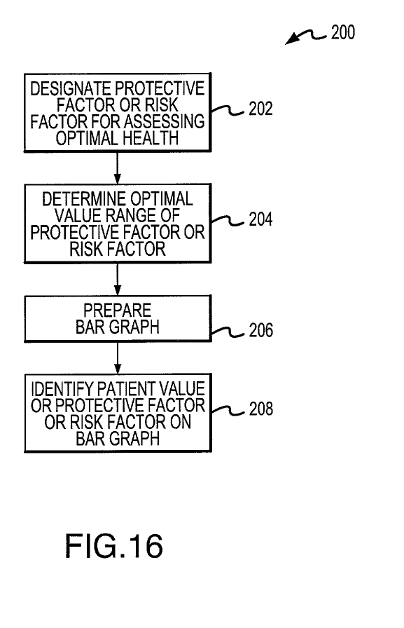

Turning now to FIG. 16, a flow chart 200 for depicting a method for profiling health status in accordance with the present invention is shown. In Step 1 202, at least one protective factor or risk factor is identified for assessing optimal health. Assessing optimal health may be performed in a number of ways including assessing cardiovascular risk, assessing oxidative stress, assessing age-related hormones, assessing trace metals, assessing liver indicator functions, assessing renal indicator functions, and depicting cancer screens. An optimal value range which represents optimal health status is then determined for the protective factor or risk factor in Step 2 204. In other words, the optimal value range for the protective factor or risk factor is based on a defined population which exhibits optimal health status as compared to a normal population range. A bar graph for profiling optimal health is then prepared in Step 3 206. The bar graph includes the optimal value range determined in Step 2 204 as well as a range of values outside of the optimal value range. Finally, in Step 4 208, a patient's or individual's value for the protective factor or risk factor designated in Step 1 202 is determined and then identified or marked on the bar graph in relation to the range of values represented by the bar graph.

After performing this method for a plurality of protective factors and risk factors related to optimal health and organizing them in column fashion, a patient or individual and physician can easily and quickly identify those protective factors and/or risk factors for a patient or individual which fall outside of the optimal value range. The patient or individual and physician can them customize a therapeutic program geared to bringing those protective factors and/or risk factors from outside of the optimal value range back into the optimal value range in order to increase the probability for the patient's or individual's optimal health.

The present invention has been described above with reference to exemplary embodiments. However, those skilled in the art having read this disclosure will recognize that changes and modifications may be made to the exemplary embodiments without departing from the scope of the present invention. These and other changes and/or modifications are intended to be included within the scope of the present invention, as expressed in the following claims.

We claim:

1. A method for profiling risk factors and protection factors comprising the steps of:
determining an optimal value range for at least one of a risk factor and a protection factor;
preparing a bar graph which includes said optimal value range and a range of values outside of said optimal value range; and
identifying a patient value for said at least one risk factor and protection factor on said bar graph.

2. The method of claim 1 wherein said optimal value range and said range of values outside of said optimal value range are represented by at least one of different colors, different symbols, and different shadings.

3. The method of claim 1 wherein said optimal value range of said at least one indicator substance is determined by an optimal mean value of a defined population exhibiting optimal cardiovascular status.

4. The method of claim 1 wherein said step of preparing a bar graph comprises the step of preparing at least one of a horizontal or vertical bar graph.

5. The method of claim 1 wherein said step of identifying a patient value of said at least one indicator on said bar graph comprises the step of marking said patient value on or near said bar graph in relation to the value ranges represented by said bar graph with at least one of a symbol, a figure, and a number.

6. The method of claim 1 wherein the patient value of said at least one indicator is determined by evaluating at least one of a blood sample and a urine sample from said patient.

7. The method of claim 1 wherein said at least one risk factor and protection factor is an indicator substance relating to at least one of cardiovascular risk, cancer risk, oxidative stress, renal function, and liver function.

8. The method of claim 1 wherein said at least one risk factor and protection factor comprises at least one of an age-related hormone and a trace element.

9. A method for profiling oxidative stress comprising the steps of:
designating at least one indicator substance for assessing oxidative stress;
determining an optimal value range of said at least one indicator which represents optimal cardiovascular status;
preparing a bar graph for profiling oxidative stress which includes said optimal value range and a range of values outside of said optimal value range; and
identifying a patient value of said at least one indicator substance on said bar graph for profiling oxidative stress.

10. The method of claim 9 wherein said optimal value range and said range of values outside of said optimal value range are represented by at least one of different colors, different symbols, and different shadings.

11. The method of claim 9 wherein said optimal value range of said at least one indicator substance is determined by an optimal mean value of a defined population exhibiting optimal cardiovascular status.

12. The method of claim 9 wherein said step of preparing a bar graph comprises the step of preparing at least one of a horizontal or vertical bar graph.

13. The method of claim 9 wherein said step of identifying a patient value of said at least one indicator on said bar graph comprises the step of marking said patient value on or near said bar graph in relation to the value ranges represented by said bar graph with at least one of a symbol, a figure, and a number.

14. The method of claim 9 wherein the patient value of said at least one indicator is determined by evaluating at least one of a blood sample and a urine sample from said patient.

15. The method of claim 9 wherein said at least one indicator substance comprises at least one of an indicator related to oxidative protection and at least one of an indicator related to oxidative damage.

16. The method of claim 15 wherein said at least one of an indicator related to oxidative protection comprises at least one indicator from the group comprising albumin, ascorbate, direct bilirubin, total bilirubin, ceruloplasmin, iron binding capacity, selenium, glutathione, ORAC, thiols, coenzyme Q10, alpha carotene, beta carotene, beta cryptoxanthin, lutein, lycopene, retinol, retinyl palmitate, zeaxanthin, alpha tocopherol, delta tocopherol, gamma tocopherol, and uric acid.

17. The method of claim 15 wherein said at least one of an indicator related to oxidative damage comprises at least one indicator from the group of copper, glucose, ferritin, fructosamine, peroxides, iron and iron binding percent saturation.

18. A method for profiling cardiovascular risk comprising the steps of:
designating at least one indicator substance for assessing cardiovascular risk;
determining an optimal value range of said at least one indicator substance which represents optimal cardiovascular status;
preparing a bar graph for profiling cardiovascular risk which includes said optimal value range and a range of values outside of said optimal value range; and
identifying a patient value of said at least one indicator substance on said bar graph for profiling cardiovascular risk.

19. The method of claim 18 wherein said optimal value range and said range of values outside of said optimal value range are represented by at least one of different colors, different symbols, and different shadings.

20. The method of claim 18 wherein said optimal value range of said at least one indicator substance is determined by an optimal mean value of a defined population exhibiting optimal cardiovascular status.

21. The method of claim 18 wherein said step of preparing a bar graph comprises the step of preparing at least one of a horizontal or vertical bar graph.

22. The method of claim 18 wherein said step of identifying a patient value of said at least one indicator substance on said bar graph comprises the step of marking said patient value on or near said bar graph in relation to the value ranges represented by said bar graph with at least one of a symbol, a figure, and a number.

23. The method of claim 18 wherein the patient value of said at least one indicator is determined by evaluating at least one of a blood sample and a urine sample from said patient.

24. The method of claim 18 wherein said at least one indicator substance comprises at least one of an indicator related to cardiovascular protection and at least one of an indicator related to cardiovascular damage.

25. The method of claim 24 wherein said at least one of an indicator related to cardiovascular protection comprises at least one indicator from the group comprising apolipoprotein A1, ascorbate, coenzyme Q10, alpha tocopherol, transferrin, ORAC, folic acid, high density lipoproteins, iron binding capacity, and vitamin B12.

26. The method of claim 24 wherein at least one of an indicator related to cardiovascular damage comprises at least one indicator from the group of apolipoprotein B, C reactive protein, cholesterol, fibrinogen, fructosamine, ferritin, homocysteine, iron, iron binding percent saturation, low density lipoproteins, lipoprotein a, a ratio of cholesterol to HDL, a ratio of LDL to HDL, a ration of apolipoprotein A1 to apolipoprotein B, and triglycerides.

27. A graphic representation for profiling at least one of oxidative stress and cardiovascular risk comprising:
a bar graph which includes an optimal value range for at least one indicator substance for assessing at least one of oxidative stress and cardiovascular risk which represents optimal cardiovascular status and a range of values outside of said optimal value range; and
a patient value for said at least one indicator substance identified on or near said bar graph in relation to the value ranges represented by said bar graph.

28. The graphic representation of claim 27 wherein said optimal value range and said range of values outside of said optimal value range are represented by at least one of different colors, different symbols, and different shadings.

29. The graphic representation of claim 27 wherein said optimal value range of said at least one indicator is determined by an optimal mean value of a defined population exhibiting optimal cardiovascular status.

30. The graphic representation of claim 27 wherein said bar graph is at least one of a horizontal bar graph or a vertical bar graph.

31. The graphic representation of claim 27 wherein the patient value of said at least one indicator is identified by at least one of a symbol, a figure, and a number.

32. The graphic representation of claim 27 wherein the patient value of said indicator is determined by evaluating at least one of a blood sample and a urine sample from said patient.

33. The graphic representation of claim 27 wherein said at least one indicator substance comprises at least one of an indicator related to oxidative protection, at least one of an indicator related to oxidative damage, at least one of an indicator related to cardiovascular protection, and at least one of an indicator related to cardiovascular damage.

34. The graphic representation of claim 33 wherein said at least one of an indicator related to oxidative protection comprises at least one indicator from the group comprising albumin, ascorbate, direct bilirubin, total bilirubin, ceruloplasmin, iron binding capacity, selenium, glutathione, ORAC, thiols, coenzyme Q10, alpha carotene, beta carotene, beta cryptoxanthin, lutein, lycopene, retinol, retinyl palmitate, zeaxanthin, alpha tocopherol, delta tocopherol, gamma tocopherol, and uric acid.

35. The graphic representation of claim 33 wherein said at least one of an indicator related to oxidative damage comprises at least one indicator from the group of copper, glucose, ferritin, fructosamine, peroxides, iron and iron binding percent saturation.

36. The graphic representation of claim 33 wherein said at least one of an indicator related to cardiovascular protection comprises at least one indicator from the group comprising apolipoprotein A1, ascorbate, coenzyme Q10, alpha tocopherol, transferrin, ORAC, folic acid, high density lipoproteins, iron binding capacity, and vitamin B12.

37. The graphic representation of claim 33 wherein at least one of an indicator related to cardiovascular damage comprises at least one indicator from the group of apolipoprotein B, C reactive protein, cholesterol, fibrinogen, fructosamine, ferritin, homocysteine, iron, iron binding percent saturation, low density lipoproteins, lipoprotein a, a ratio of cholesterol to HDL, a ratio of LDL to HDL, a ration of apolipoprotein A1 to apolipoprotein B, and triglycerides.
